(19) United States
(12) Patent Application Publication
(10) Pub. No.: US 2004/0157855 A1
(43) Pub. Date: Aug. 12, 2004

(54) Use of n-phenyl-2-pyrimidineamine derivativea against mast cell-based diseases like allergic disorders

(76) Inventors: Michael Heinrich, Lake Oswego, OR (US); Kenichi Koike, Nagano Pref (JP); Atsushi Komiyama, Nagano (JP); Motowo Nakajima, Hyogo (JP); Kouichi Takeuchi, Nagano (JP)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/473,792

(22) PCT Filed: Apr. 5, 2002
(86) PCT No.: PCT/US02/10742

(30) Foreign Application Priority Data
Apr. 5, 2001 (GB) 0108606.5

(51) Int. Cl. A61K 31/506; C 07D 4 3/02

(52) U.S. Cl. 514/252.14; 544/295; 544/331; 514/275

(57) ABSTRACT

Use of the N-phenyl-2-pyrimidine-amine derivatives of formula I, in which the symbols and substituents have the meaning as given herein in free form or in pharmaceutically acceptable salt form in the manufacture of a pharmaceutical composition for the treatment of allergic rhinitis, allergic dermatitis, drug allergy or food allergy, angioedema, urticaria, sudden infant death syndrome, bronchopulmonary aspergillosis, multiple sclerosis or mastocytosis.

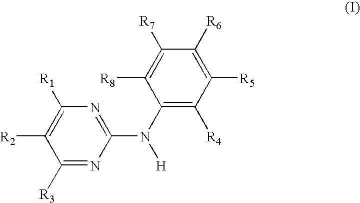

The present invention relates to a new use of the N-phenyl-2-pyrimidine-amine derivatives of formula I in which the symbols and substituents have the meaning as given hereinafter in free form or in pharmaceutically acceptable salt form in the manufacture of a pharmaceutical composition for the treatment of allergic rhinitis, allergic dermatitis, drug allergy or food allergy, angioedema, urticaria, sudden infant death syndrome, bronchopulmonary aspergillosis, multiple sclerosis or mastocytosis; and to a method of treatment of warm-blooded animals, preferably humans, in which a therapeutically effective dose of a compound of formula I is administered to a warm-blooded animal suffering from one of the diseases mentioned above.

The present invention relates the use of N-phenyl-2-pyrimidine-amine derivatives of formula I

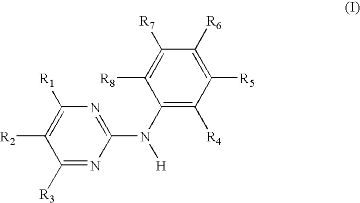

wherein

R₁ is 4-pyrazinyl; 1-methyl-1H-pyrrolyl; amino- or amino-lower alkyl-substituted phenyl, wherein the amino group in each case is free, alkylated or acylated; 1H-indolyl or 1H-imidazolyl bonded at a five-membered ring carbon atom; or unsubstituted or lower alkyl-substituted pyridyl bonded at a ring carbon atom and unsubstituted or substituted at the nitrogen atom by oxygen;

R₂ and R₃ are each independently of the other hydrogen or lower alkyl;

one or two of the radicals R₄, R₅, R₆, R₇ and R₈ are each nitro, fluoro-substituted lower alkoxy or a radical of formula II

—N(R₉)—C(═X)—(Y)ₙ—R₁₀ (II),

wherein

R₉ is hydrogen or lower alkyl,

X is oxo, thio, imino, N-lower alkyl-imino, hydroximino or O-lower alkyl-hydroximino,

Y is oxygen or the group NH,

n is 0 or 1 and

R₁₀ is an aliphatic radical having at least 5 carbon atoms, or an aromatic, aromatic-aliphatic, cycloaliphatic, cycloaliphatic-aliphatic, heterocyclic or heterocyclic-aliphatic radical,

and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,

or of a salt of such a compound having at least one salt-forming group,

for the manufacture of a medicament for treating allergic rhinitis, allergic dermatitis, drug allergy or food allergy, angioedema, urticaria, sudden infant death syndrome, bronchopulmonary aspergillosis, mastocytosis or multiple sclerosis.

1-methyl-1H-pyrrolyl is preferably 1-methyl-1H-pyrrol-2-yl or 1-methyl-1H-pyrrol-3-yl.

Amino- or amino-lower alkyl-substituted phenyl R₁ wherein the amino group in each case is free, alkylated or acylated is phenyl substituted in any desired position (ortho, meta or para) wherein an alkylated amino group is preferably mono- or di-lower alkylamino, for example, dimethylamino, and the lower alkyl moiety of amino-lower alkyl is preferably linear C₁-C₃alkyl, such as especially methyl or ethyl.

1H-indolyl bonded at a carbon atom of the five-membered ring is 1H-indol-2-yl or 1H-indol-3-yl.

Unsubstituted or lower alkyl-substituted pyridyl bonded at a ring carbon atom is lower alkyl-substituted or preferably unsubstituted 2-, 4- or preferably 3-pyridyl, for example 3-pyridyl, 2-methyl-3-pyridyl or 4-methyl-3-pyridyl. Pyridyl substituted at the nitrogen atom by oxygen is a radical derived from pyridine N-oxide, i.e., N-oxido-pyridyl.

Fluoro-substituted lower alkoxy is lower alkoxy carrying at least one, but preferably several, fluoro substituents, especially trifluoromethoxy or 1,1,2,2-tetrafluoro-ethoxy.

When X is oxo, thio, imino, N-lower alkyl-imino, hydroximino or O-lower alkyl-hydroximino, the group C═X is, in the above order, a radical C═O, C═S, C═N—H, C═N-lower alkyl, C═N—OH or C═N—O-lower alkyl, respectively. X is preferably oxo.

n is preferably 0, i.e. the group Y is not present.

Y, if present, is preferably the group NH.

The term “lower” within the scope of this text denotes radicals having up to and including 7, preferably up to and including 4 carbon atoms.

Lower alkyl R₁, R₂, R₃ and R₉ is preferably methyl or ethyl.

An aliphatic radical R₁₀ having at least 5 carbon atoms preferably has not more than 22 carbon atoms, generally not more than 10 carbon atoms, and is such a substituted or preferably unsubstituted aliphatic hydrocarbon radical, that is to say such a substituted or preferably unsubstituted alkynyl, alkenyl or preferably alkyl radical, such as C₅-C₇alkyl, for example n-pentyl. An aromatic radical R₁₀ has up to 20 carbon atoms and is unsubstituted or substituted, for example, in each case unsubstituted or substituted naphthyl, such as especially 2-naphthyl, or preferably phenyl, the substituents preferably being selected from cyano, unsubstituted or hydroxy-, amino- or 4-methyl-piperazinyl-substituted lower alkyl, such as especially methyl, trifluoromethyl, free, etherified or esterified hydroxy, free, alkylated or acylated amino and free or esterified carboxy. In an aromatic-aliphatic radical R₁₀ the aromatic moiety is as defined above and the aliphatic moiety is preferably lower alkyl, such as especially C₁-C₂alkyl, which is substituted or preferably unsubstituted, for example benzyl. A cycloaliphatic radical R₁₀ has especially up to 30, more especially up to 20, and most especially up to 10 carbon atoms, is mono- or poly-cyclic and is substituted or preferably unsubstituted, for example, such a cycloalkyl radical, especially such a 5- or 6-membered cycloalkyl radical, such as preferably cyclohexyl. In a cycloaliphatic-aliphatic radical R₁₀ the cycloaliphatic moiety is as defined above and the aliphatic moiety is preferably lower alkyl, such as especially C₁-C₂alkyl, which is substituted or preferably unsubstituted. A heterocyclic radical R₁₀ contains especially up to 20 carbon atoms and is preferably a saturated or unsaturated monocyclic radical having 5- or 6-ring members and 1-3 hetero atoms which are preferably selected from nitrogen, oxygen and sulfur, especially, for example, thienyl or 2-, 3- or 4-pyridyl, or a bi- or tri-cyclic radical wherein, for example, one or two benzene radicals are annellated (fused) to the mentioned monocyclic radical. In a heterocyclic-aliphatic radical R₁₀ the heterocyclic moiety is as defined above and the aliphatic moiety is preferably lower alkyl, such as especially C₁-C₂alkyl, which is substituted or preferably unsubstituted.

Etherified hydroxy is preferably lower alkoxy. Esterified hydroxy is preferably hydroxy esterified by an organic carboxylic acid, such as a lower alkanoic acid, or a mineral acid, such as a hydrohalic acid, for example, lower alkanoyloxy or especially halogen, such as iodine, bromine or especially fluorine or chlorine.

Alkylated amino is, for example, lower alkylamino, such as methylamino, or di-lower alkylamino, such as dimethylamino. Acylated amino is, for example, lower alkanoylamino or benzoylamino.

Esterified carboxy is, for example, lower alkoxycarbonyl, such as methoxycarbonyl.

A substituted phenyl radical may carry up to 5 substituents, such as fluorine, but especially in the case of relatively large substituents is generally substituted by only from 1-3 substituents. Examples of substituted phenyl that may be given special mention are 4-chloro-phenyl, pentafluoro-phenyl, 2-carboxy-phenyl, 2-methoxy-phenyl, 4-fluoro-phenyl, 4-cyano-phenyl and 4-methyl-phenyl.

Salt-forming groups in a compound of formula I are groups or radicals having basic or acidic properties. Compounds having at least one basic group or at least one basic radical, for example, a free amino group, a pyrazinyl radical or a pyridyl radical, may form acid addition salts, for example, with inorganic acids, such as hydrochloric acid, sulfuric acid or a phosphoric acid, or with suitable organic carboxylic or sulfonic acids, for example, aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example, benzene-, p-toluene- or naphthalene-2-sulfonic acid. When several basic groups are present mono- or poly-acid addition salts may be formed.

Compounds of formula I having acidic groups, for example, a free carboxy group in the radical R₁₀, may form metal or ammonium salts, such as alkali metal or alkaline earth metal salts, for example, sodium, potassium, magnesium or calcium salts, or ammonium salts with ammonia or suitable organic amines, such as tertiary monoamines, for example, triethylamine or tri-(2-hydroxyethyl)-amine, or heterocyclic bases, for example, N-ethyl-piperidine or N,N′-dimethyl-piperazine.

Compounds of formula I having both acidic and basic groups can form internal salts.

For the purposes of isolation or purification, as well as in the case of compounds that are used further as intermediates, it is also possible to use pharmaceutically unacceptable salts. Only pharmaceutically acceptable, non-toxic salts are used for therapeutic purposes, however, and those salts are therefore preferred.

The compounds of formula I can be used for the treatment or for the manufacture of medicaments for the treatment of warm-blooded animals, preferably humans.

The term “allergic rhinitis” as used herein means any allergic reaction of the nasal mucosa. Such allergic reaction may occur, e.g., perennially, e.g., vernal conjunctivitis, or seasonally, e.g., hay fever.

The term “allergic dermatitis” as used herein means especially atopic dermatitis, allergic contact dermatitis and eczematous dermatitis, but comprises, e.g., also seborrhoeic dermatitis, Lichen planus, urticaria and acne. Atopic dermatitis as defined herein is a chronic inflammatory skin disorder seen in individuals with a hereditary predisposition to a lowered cutaneous threshold to pruritus. It is principally characterized by extreme itching, leading to scratching and rubbing that in turns results in the typical lesions of eczema. Allergic contact dermatitis as defined herein is a form of dermatitis that is due to the allergic sensitization to various substances that produce inflammatory reactions in the skin of those who have acquired hypersensitivity to the allergen as a result of previous exposure to it.

The term “drug allergy or food allergy” as used herein pertains to an allergic reaction produced by a drug or ingested antigens, such as, for example, strawberries, milk or eggs.

The term “bronchopulmonary aspergillosis” relates to an infection of the lungs with Aspergillus.

The term “mastocytosis” as used herein, relates to systemic mastocytosis, for example, mastocytoma, and particularly to canine mast cell neoplasms.

Compounds of formula I are being referred to hereinafter collectively as COMPOUNDS OF THE INVENTION.

Mast cells play an important role as the primary effector cells in the allergic disorders mentioned herein. Antigen-specific IgE-mediated degranulation of mast cells leads to the subsequent release of chemical mediators and multiple cytokines and to leukotriene synthesis. Furthermore, mast cells are involved in the pathogenesis of multiple sclerosis.

Mast cell neoplasms occur in both humans and animals. In dogs, mast cell neoplasms are called mastocytomas, and the disease is common, representing 7-21% of canine tumors. A distinction must be drawn between human mastocytosis, which is usually transient or indolent, and canine mast cell neoplasia, which behaves unpredictably and is often aggressive and metastatic. For instance, human solitary mastocytomas essentially never metastasize; in contrast, 50% of canine mastocytomas behave in a malignant fashion, as estimated by Hottendorf & Nielsen (1969) after review of 46 published reports of tumors in 938 dogs.

Cancer in the pet population is a spontaneous disease. Pet owners, motivated by prolonging the quality of their animals' life, frequently seek out the specialized care and treatment of veterinary oncologists at private referral veterinary hospitals and veterinary teaching hospitals across the country. Therapeutic modalities of veterinary cancer patients are similar to humans, including surgery, chemotherapy, radiation therapy, and biotherapy. It has been estimated that there are 42 million dogs and approximately 20 million cats in the United States. Using crude estimates of cancer incidence, there are roughly 4 million new cancer diagnoses made in dogs and a similar number in cats made each year.

Cutaneous mast cell tumors in dogs are a common problem. Most mast cell tumors are benign and are cured with simple resection; however, if recurrent or metastatic to distant sites therapeutic options are limited. Treatment options for recurrent lesions can include external beam radiation therapy. For distant metastases or disseminated disease the use of Lomustine® and vinblastine containing chemotherapy protocols have demonstrated some benefit. Sites for metastases for mast cell tumors include skin, regional lymph nodes, spleen, liver and bone marrow.

The KIT receptor's involvement in the pathogenesis of mastocytosis is suggested by the observation that several mutations resulting in constitutive activation of KIT have been detected in a number of mast cell lines. For instance, a point mutation in human c-KIT, causing substitution of Val for Asp816 in the phosphotransferase domain and receptor autoactivation, occurs in a long-term human mast cell leukemia line (HMC-1) and in the corresponding codon in two rodent mast cell lines. Moreover, this activating mutation has been identified in situ in some cases of human mastocytosis. Two other activating mutations have been found in the intracellular juxtamembrane region of KIT, i.e., the Val560Gly substitution in the human HMC-1 mast cell line, and a seven amino acid deletion (Thr573-His579) in a rodent mast cell line called FMA3.

It can be shown by established test models and especially those test models described herein that the COMPOUNDS OF THE INVENTION or in each case a pharmaceutically acceptable salt thereof, results in an effective prevention or, preferably, treatment of at least one of the diseases mentioned herein. The person skilled in the pertinent art is fully enabled to select a relevant test model to prove the hereinbefore and hereinafter indicated therapeutic indications and beneficial effects. The pharmacological activity may, for example, be demonstrated in a clinical study or in the test procedure as essentially described hereinafter.

Part A

### EXAMPLE 1

This example demonstrates the in vitro effects of the COMPOUNDS OF THE INVENTION on the SCF-dependent development of cultured human mast cell growth generated from CD34⁺ cord blood cells using the culture system described by Kinoshita T, Sawai N, et al. in Blood, Vol. 94, pp. 496-508 (1999). More than 90% of the isolated cells were CD34-positive according to the flow cytometric analysis.

Reagents and Antibodies

The COMPOUNDS OF THE INVENTION are solubilized in PBS at a concentration of 10⁻² M and stored at −80° C. All-trans retinoic acid (Sigma) is dissolved in ethanol at a concentration of 10⁻² M, and stored in light-protected vials at −80° C. Purified mAb for tryptase (MAB1222) can be purchased from Chemicon International Inc., CA. For the flow cytometric analysis, the mAbs for CD34 (8G12, FITC) and CD11b (Leu15, PE) are purchased from Becton Dickinson Immunocytometry Systems (Mountain View, Calif.), and the mAb for CD41 (SZ22, FITC) from Immunotech S.A. (Marseilles, France). The mAb for glycophorin A (GPA, JC159, FITC) can be obtained from Dako (Glostrup, Denmark). For western blotting and immunoprecipitation, the mAbs for c-kit (NU-c-kit) and for phosphotyrosine (4G10) can be purchased from Nichirei and Upstate Biotechnology, Inc (Lake Placid, N.Y.), respectively.

Suspension Cultures

Serum-deprived liquid cultures are carried out in 24-well culture plates (#3047; Becton Dickinson). Twenty thousand CD34⁺ cells are cultured in each well containing 2 mL of α-medium supplemented with 1% BSA, 300 μg/mL fully iron-saturated human transferrin (approximately 98% pure, Sigma), 16 μg/mL soybean lecithin (Sigma), 9.6 μg/mL cholesterol (Nakalai Chemicals Ltd., Japan) and 20 ng/mL of SCF, 10 ng/mL of GM-CSF, 2 U/mL of EPO, 10 ng/mL of TPO, different concentrations of a COMPOUND OF THE INVENTION, alone or in combination. In order to examine the effects of a COMPOUND OF THE INVENTION on the SCF-dependent development of mast cells, 10-week cultured cells grown with 20 ng/mL of SCF from CD34⁺ cord blood cells are used as target cells. Five to ten×10⁴ 10-week cultured cells are incubated for 2 weeks in 24-well culture plates containing 20 ng/mL of SCF with or without various concentrations of a COMPOUND OF THE INVENTION. The plates are incubated at 37° C. in a humidified atmosphere flushed with a mixture of 5% CO₂, 5% O₂ and 90% N₂. When the culture continued until 4 weeks, half of the culture medium is replaced every 2 weeks with fresh medium containing the factor(s). The number of viable cells is determined by a trypan-blue exclusion test using a hemocytometer.

Clonal Cell Cultures

The mast cell colony assay is carried out in 35 mm Lux suspension culture dishes (#171099; Nunc, Ill.). The culture consisted of 10-week cultured cells (4,000 cells/mL) grown with 10 ng/mL of SCF, α-medium, 0.9% methylcellulose (Shinetsu Chemical, Japan),1% BSA, 300 μg/mL of fully iron-saturated human transferrin, 16 μg/mL of soybean lecithin, 9.6 μg/mL of cholesterol and 100 ng/mL of SCF with or without 10⁻⁶ M of a COMPOUND OF THE INVENTION. Dishes are incubated at 37° C. in a humidified atmosphere flushed with a mixture of 5% CO₂, 5% O₂ and 90% N₂. After 4 weeks, aggregates consisting of 30 or more cells are scored as mast cell colonies, and those consisting of 10-29 cells as mast cell clusters. Thirty individual colonies and clusters are lifted, and stained with the anti-tryptase mAb or mouse IgG1 using the alkaline phosphatase-anti-alkaline phosphatase (APAAP) technique. Almost all of the constituent cells are positive for tryptase.

Cytochemical and Immunologic Stainings

The cultured cells are spread on glass slides using a Cytospin II. Cytochemical reaction with peroxidase (POX) is performed by the conventional method. Reaction with mAb against tryptase is detected using the APAAP method (Dako APAAP Kit System, Dako Corp., CA), as described by Ma et al., Br. J. Haematol., Vol. 100, pp. 427435 (1998).

Immunoprecipitation and Western Blotting

Immunoprecipitation and Western blotting are performed, as described by Kamijo et al., Leuk. Res., Vol. 21, pp 1097-1106 (1997).

Flow cytometric Analysis

For the analysis of surface markers on the cultured cells, 1-2×10⁵ cells are collected in plastic tubes and incubated with appropriately diluted FITC- or PE-mAb, as described by Kinoshita et al., Blood, Vol. 94, pp. 496-508 (1999). The cells are washed twice, after which their surface markers are analyzed with the FACScan flow cytometer. Viable cells are gated according to their forward light scatter characteristics and side scatter characteristics. The proportion of positive cells is determined by comparison to cells stained with FITC- or PE-conjugated mouse isotype-matched Ig.

Detection of Cellular Apoptosis

The analysis of cellular apoptosis is carried out by a flow cytometric analysis using propidium iodide (PI, Sigma) according to the procedure described by Sawai et al., Stem Cells, Vol. 17, pp. 45-53 (1999). In order to reduce cells undergoing apoptosis, necrosis or already dead, a percoll gradient centrifugation can be utilized. Ten-week cultured cells are layered on 27% Percoll (Sigma) in α-medium and 54% Percoll in PBS. After centrifugation, the cells are collected from the interface of the two different concentrations of Percoll solution, washed with PBS and treated with 1 mL of Ortho PermeaFix™ for 40 minutes at room temperature. The cells are then incubated with DNase-free RNase (Sigma) for 15 minutes at 37° C., and stained with PI for 15 minutes. The DNA content of 2×10⁴ cells is monitored with a flow cytometer.

The 10-week cultured cells (2×10⁶) exposed to SCF or SCF and a COMPOUND OF THE INVENTION are lysed for 10 minutes on ice in 100 μL hypotonic lysis buffer [10 mM Tris (pH 7.5), 10 mM EDTA, pH 8.0, 0.5% Triton X-100]. After centrifugation for 10 minutes at 14,000 g, the supernatant is transferred to a new tube, and treated with 0.2 mg/mL RNase A (Sigma) and 0.2 mg/mL Proteinase K (Sigma). DNA is precipitated with 120 μL isopropanol and 20 μL 5 M NaCl overnight at −20° C. After centrifugation at 14,000 g, the pellets are dried, dissolved in 20 μL Tris-EDTA, and then samples are analyzed by gel electrophoresis in 2% agarose and ethidium bromide staining.

Assay of Histamine, Tryptase and Cytokine Levels

Histamine concentrations in cell lysates obtained by the treatment of the cultured cells with 0.5% Nonidet P40 and in supernatant are measured by Histamine Radioimmunoassay (RIA) Kit (Immunotech), as described in Kinoshita et al., Blood, Vol. 94, pp. 496-508 (1999).

Statistical Analysis

All experiments should be carried out at least three times. To determine the significance of difference between two independent groups, the unpaired t-test can be used, orthe Mann-Whitney-U test when the data are not normally distributed.

Results as Obtained for N-{5-[4-(4-methyl-piperazino-methyl)-benzoylamido]-2-methyl-phenyl}-4-(3-pyridyl)-2-pyrimidine-amine monomesylate (STI571B)

Addition of STI571B at 10⁻⁶ M or higher to the culture with SCF almost completely inhibits the progeny generation.

In SCF plus STI571B, the number of viable cells is unchanged on day 2 and then declines in a time-dependent fashion. On day 14, the total cell number reaches to a negligible level. Consistent with the results obtained by 10-week cultured mast cells, STI571B at 10⁻⁶ M markedly depresses the cell production by CD34⁺ cells under stimulation with SCF. Furthermore, STI571B induces a programmed death of the cultured mast cells grown under stimulation with SCF. The percentage of sub-diploid nuclei increases with the culture period. This observation can be confirmed by DNA laddering in cells exposed to SCF plus STI571B on gel electrophoresis. STI571B exerts its inhibitory effects at the early phase of mast cell development as well as the growth of the late-appearing mast cells.

The obtained results demonstrate the inhibition of the SCF-dependent human mast cell growth by COMPOUNDS OF THE INVENTION, e.g., STI571B. Hence, it is shown that the COMPOUNDS OF THE INVENTION can be used for the treatment of the diseases mentioned herein.

Part B: Mastocytosis

### EXAMPLE 2

### Methods

Reagents: Novartis Pharma; Basel, Switzerland provided SALT I for use in these experiments. Fresh 10 mM stock solutions of the inhibitor were made before each experiment by dissolving compound in 1 mL Phosphate-Buffered Saline (PBS; Gibco-BRL).

Antibodies: A polyclonal rabbit anti-KIT antibody (c-kit Ab-1) was used at a dilution of 1:500 (c-kit Ab-1; Oncogene, Cambridge, Mass.). An anti-phosphotyrosine antibody (PY20) was used at a dilution of 1:1000 (PY20 Transduction Laboratories; Lexington, K.Y.). Peroxidase conjugated goat anti-mouse antibody was used at a dilution of 1:5000 and goat anti-rabbit antibody at a dilution of 1:10,000 (Pierce; Rockford, Ill.).

Cell lines: BR and C2 canine mastocytoma cells lines were obtained from Dr. George Caughey (University of California at San Francisco, San Francisco, Calif.). Both cell lines were maintained in DMEM supplemented with 2% bovine calf, 1 mM L-glutamine, 12.5 mM HEPES (pH 7.5), 0.25 mg/ml Histidine, 1 % Penicillin-Streptomycin and 1% fungizone. The BR and C2 cells were derived from canine mast cell tumors and were originally established in long-term culture after initial passaging in immunodeficient mice (DeVinney R et al., Am J Respir Cell Mol Biol 1990; 3(5):413-420; Lazarus SC et al., Am J Physiol 1986; 251(6 Pt 1):C935-C944). The BR cell line has a point mutation (T1752C) resulting in a Leucine to Proline substitution at amino acid 575 (juxtamembrane domain). The C2 cell line has an internal tandem duplication (ITD) of the KIT juxtamembrane region. The translation of this ITD results in reduplication of amino acid residues at the 3′ end of exon 11 (London et al., Exp. Hematol., Vol. 27, No. 4, pp. 689-697 (1999); Ma et al., J. Invest. Dermatol., Vol. 112, No. 2, pp. 165-170 (1999)).

Proliferation assays: Cells were added to 96-well plates at a density of 40,000 cells/well in normal culture media and varying concentrations of SALT I. Proliferation was measured at 48-72 hours using an XTT-based assay (Roche Molecular Biochemicals; Indianapolis, Ind.) (Heinrich et al., Blood, Vol. 96, No. 3, pp. 925-932 (2000))

Protein lysates: BR and C2 cells were washed×2 in PBS and then quiesced in Optimem (Gibco-BRL) at 37° C. for approximately 18 hours. Cells were then incubated for 90 minutes in the presence of various concentrations of SALT I. Following this incubation, the cells were pelleted and lysed using 100-250 μL of protein lysis buffer (50 mM Tris, 150 mM NaCl, 1% NP-40, 0.25% Deoxycholate, with addition of the inhibitors aprotinin, leupeptin, pepstatin, PMSF and sodium orthovanadate [Sigma]). Western immunoblot analysis was performed as previously described (Hoatlin et al., Blood, Vol. 91, No. 4, pp. 1418-1425 (1998); Heinrich et al., Blood, Vol. 96, No. 3, pp. 925-932 (2000)).

### EXAMPLE 3

### COMPOUND I Inhibits the Constitutively Activated KIT Kinase Associated with Canine Mast Cell Tumors

To test the efficacy of COMPOUND I in inhibiting the kinase activity of mutant forms of canine KIT we used two cells lines (BR and C2) that express two different constitutively activated KIT isoforms. The KIT mutations in these cell lines are both located in the juxtamembrane domain and are homologous to mutations seen in human Gastrointestinal Stromal Tumors (GISTs) (Lux et al., Am. J. Pathol., Vol. 156, No. 3, pp. 791-795 (2000); Rubin et al., Cancer Res., Vol. 61, No. 22, pp. 8118-8121 (2001)). Lysates prepared from BR or C2 cells were probed with an anti-P-Tyr antibody and KIT receptor activation was assessed by measuring autophosphorylation. As reported previously, KIT autophosphorylation in these cells was observed in the absence of SLF (Ma et al., J. Invest. Dermatol., Vol. 112, No. 2, pp. 165-170 (1999); Ma et al., J. Invest. Dermatol., Vol. 114, No. 2, pp. 392-394 (2000)). Inhibition of KIT autophosphorylation by COMPOUND I was dose dependent with complete inhibition observed using 10 and 1.0 μM doses. Near complete inhibition was seen using a dose of 0.1 μM. Limited autophosphorylation of c-kit was seen using 0.001-0.01 μM doses of COMPOUND I. Thus, COMPOUND I not only inhibits the autophosphorylation of the mutated c-kit receptor in these cells, but also is a more potent inhibitor of this mutated receptor than it is of the wild type c-kit receptor (IC₅₀ 100-200 nM) (Heinrich et al., Blood, Vol. 96, No. 3, pp. 925-932 (2000)). To determine if COMPOUND I modulated expression of KIT protein, the membrane was stripped and reprobed with an anti-c-kit antibody. There was no change in expression of c-kit protein in of COMPOUND I treated cells. Therefore, COMPOUND I decreases autophosphorylation of mutant canine KIT polypeptide by inhibiting KIT kinase activity rather than by down regulating expression of KIT protein.

### EXAMPLE 4

### COMPOUND I Inhibits the Proliferation of Cell Lines of Canine Mast Cell Tumors

To test the biologic effect of inhibiting the kinase activity of a mutant c-kit receptor, we cultured BR or C2 cells for 48-72 hours in the presence of various concentrations of COMPOUND I. At inhibitor concentrations of 0.1-10 μM, proliferation was decreased by 90-95% compared to cells treated with media only. Partial inhibition of proliferation was seen at doses of 0.001-0.01 μM COMPOUND I. The decrease in proliferation seen with doses of 0.01-10 μM inhibitor was statistically significant (p<0.001). Therefore, COMPOUND I inhibits proliferation of BR and C2 cells with the same dose response range as seen for inhibition of receptor autophosphorylation. Morphologic observations of the inhibitor treated cells revealed changes consistent with apoptosis (data not shown).

TABLE 1
BR cells

| | | Averages | % | SD | % SD |
|---|---|---|---|---|---|
| Cells | | 0.929 | 100% | 0.030447 | 3% |
| 5 | μM | 0.083 | 9% | 0.001732 | 0% |
| 1 | μM | 0.105 | 11% | 0.002 | 0% |
| .1 | μM | 0.105 | 11% | 0.002082 | 0% |
| .01 | μM | 0.479 | 52% | 0.043016 | 5% |
| .001 | μM | 0.781 | 84% | 0.033081 | 4% |

TABLE 2
C2 cells

| | | Averages | % | SD | % SD |
|---|---|---|---|---|---|
| Cells | | 1.236 | 100% | 0.04417 | 4% |
| 5 | μM | 0.032 | 3% | 0.005686 | 0% |
| 1 | μM | 0.037 | 3% | 0.013868 | 1% |
| .1 | μM | 0.028 | 2% | 0.003512 | 0% |
| .01 | μM | 0.754 | 61% | 0.185236 | 15% |
| .001 | μM | 1.065 | 86% | 0.055245 | 4% |

Tables 1 and 2: BR or C2 cells were plated in 96-well plates at a concentration of 40,000 cells/well and cultured in normal growth media and varying concentration of COMPOUND I. Cellular proliferation was measured at 72 hours using an XTT-based assay system. Each COMPOUND I concentration was assayed in triplicate. Results are expressed as a percent of maximal proliferation (cells only, no COMPOUND I)±1 standard deviation. Representative results from one of six independent experiments are shown.

Compositions for Pets and Humans

### EXAMPLE 5

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-crystal form

Capsules containing 11.95 mg of the compound named in the title (=SALT I) corresponding to 10.0 mg of COMPOUND I (free base) as active substance are prepared in the following composition:

| Composition | | |
|---|---|---|
| SALT I | 11.95 | mg |
| Cellulose MK GR | 9.2 | mg |
| Crospovidone XL | 1.5 | mg |
| Aerosil 200 | 0.2 | mg |
| Magnesium stearate | 0.15 | mg |
| | 23.0 | mg |

The capsules are prepared by mixing the components and filling the mixture into hard gelatin capsules, size 1.

### EXAMPLE 6

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-crystal form

Capsules containing 10.0 mg of the compound named in the title (=SALT I) as active substance are prepared in the following composition:

| Composition | | |
|---|---|---|
| Active substance | 10.0 | mg |
| Avicel | 20.0 | mg |
| PVPPXL | 1.5 | mg |
| Aerosil | 0.2 | mg |
| Magnesium stearate | 0.15 | mg |
| | 31.85 | mg |

The capsules are prepared by mixing the components and filling the mixture into hard gelatin capsules, size 1.

### EXAMPLE 7

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-crystal form.

Capsules containing 119.5 mg of the compound named in the title (=COMPOUND I mesylate) corresponding to 100 mg of COMPOUND I (free base) as active substance are prepared in the following composition:

| Composition | | |
|---|---|---|
| COMPOUND I mesylate | 119.5 | mg |
| Cellulose MK GR | 92 | mg |
| Crospovidone XL | 15 | mg |
| Aerosil 200 | 2 | mg |
| Magnesium stearate | 1.5 | mg |
| | 230 | mg |

The capsules are prepared by mixing the components and filling the mixture into hard gelatin capsules, size 1.

### EXAMPLE 8

### Example of a Prospective Case Series of Pet Dogs with Measurable Cutaneous Mast Cell Tumors.

The study patients are pet dogs with measurable and histologically-confirmed mast cell tumors. Cases are limited to those with measurable lesions amenable to biopsy.

Eligibility criteria are:

Histologically-confirmed measurable cutaneous mast cell tumors

Cases will require serial biopsy with 2 mM Keyes punch before and during therapy

Histological grade (II-intermediate or III-poorly differentiated)

Performance status 0 or I (Modified Karnofsky—Table 3)

Informed owner consent

Exclusion criteria are:

Concurrent cytotoxic chemotherapy

Prednisone and non-steroidal anti-inflammatory drugs may not be initiated within 30 days of the study; if prednisone or non-steroidal anti-inflammatory drugs have been administered for greater than 30 days they may be continued

Abnormal serum bile acid test (liver function)

TABLE 3
Performance Status (Modified Karnofsky)

| Grade | Description |
|---|---|
| 0 | Normal activity |
| 1 | Restricted activity; decreased activity from pre-disease |
| | status |
| 2 | Compromised; ambulatory only for vital activities; |
| | consistently defecates and urinates in acceptable areas |
| 3 | Disabled; must be force fed; unable to confine urination |
| | and defecation to acceptable areas |
| 4 | Dead |

Pretreatment evaluation of all cases include physical examination, complete blood count, buffy coat, serum biochemistry, urinalysis, serum bile acids (fasting and post-prandial), documentation of regional lymph node size, abdominal radiographs, and abdominal ultrasound. The treatment regimen is 25 mg/kg PO QD×60 days of SALT I.

Treatment is continued in all cases for 60 days unless disease progression is noted. In cases experiencing partial response or complete response ongoing therapy for an additional 60 days may be considered. Cases successfully completing therapy are eligible for repeat entry to study.

TABLE 4
Treatment and Clinical Evaluation Plan

| Action | Day 0 | Day 7 | Day 14 | Day 28 | q14 days |
|---|---|---|---|---|---|
| Clinical staging¹ | X | | | X | X |
| Physical examination | X | X | X | X | X |
| Measurement of | X | X | X | X | X |
| tumor burden² | | | | | |
| Start SALT I | X | | | | |
| 25 mg/kg QD | | | | | |
| Pharmacokinetics³ | X | | | | |
| Incisional biopsy⁴ | X | | | X | |
| Repeat Staging | | | | X | |

¹Initial staging consists of physical examination, CBC, buffy coat, serum biochemistry, liver function tests (serum bile acids), urinalysis, abdominal radiographs, and abdominal ultrasound. Re-evaluation of may consist of physical examination and measurement of tumor burden alone or repeat clinical staging.
²Tumor burden is measured at days 0, 7, 14 and 28, and then every 14 days. Treatment response will be defined against measurable cutaneous lesion(s) and other lesions identified at staging (CR, PR, SD, PD - defined below).
³Collection of plasma from the first 5 entered cases is undertaken at 0, 0.5, 1, 2, 5, 8, 12, 16 and 24 hours following first dose of SALT I.
⁴Incisional biopsy from defined measurable lesion(s) will be collected on days 0 and 28 from all cases. Additional biopsies are collected at the time of PR and after objective CR.

The efficacy of COMPOUND I is assessed against measurable cutaneous mast cell tumors, using clinical endpoints. Biological endpoints may be taken from serial biopsies collected from cutaneous tumors and from blood samples available through the treatment course.

Clinical endpoints include response rate of measurable tumors, objective response against measurable tumor, and time to progression of measurable tumor. All adverse side effects will be recorded.

“Objective Tumors Responses”, as defined below, are observed under treatment with COMPOUND I and indicate efficacy of the treatment regimen.

In particular, Complete Responses (CR) and Partial Responses (PR) to treatment with COMPOUND I may be observed. Furthermore, it may be observed that more animals obtaining treatment show Stable Disease (SD), while less treated animals show Progressive Disease (PD). Also, it may be observed that less animals obtaining treatment show Relapse (R) of disease as compared to non-treated animals. Time To Progression (TTP), Duration of Remission and Survival may increase in animals under treatment with COMPOUND I.

“CR” is defined as disappearance of all clinical evidence of cancer and of any signs related to the cancer.

“PR” is defined as a 50% or greater decrease in the sum of the products of measurements for representative lesions, without an increase in size of any lesions or appearance of any new lesions.

“SD” is defined as no response or a response of less than that defined for partial response or progressive disease without appearance of any new lesions or worsening of clinical signs.

“PD” is defined as an unequivocal increase of at least 50% in the size of any measurable lesion or appearance of new lesions.

“R” is defined as appearance of new lesions or reappearance of old lesions in dogs that had had a CR; in dogs that had had only a PR, R was defined as at least a 50% increase in the sum of the products of measurements of representative lesions, compared with measurements obtained at the time of maximum response.

“TTP” is reported from day 0 of the protocol. TTP will be defined as the number of days start of therapy (from day 0) to R.

“Duration of Remission” is defined as the number of days from the objective response (PR or CR) to R.

“Survival” is defined as the number of days from the start of treatment with COMPOUND I to death. Cause of death will be noted but may include disease progression, toxicity and other.

The obtained results demonstrate the inhibition of the mast cell neoplasms by COMPOUNDS OF THE INVENTION, e.g., STI571B.

Preference is given to COMPOUNDS OF THE INVENTION wherein

one or two of the radicals R₄, R₅, R₆, R₇ and R₈ are each nitro or a radical of formula II wherein R₉ is hydrogen or lower alkyl, X is oxo, thio, imino, N-lower alkyl-imino, hydroximino or O-lower alkyl-hydroximino, Y is oxygen or the group NH, n is 0 or 1 and R₁₀ is an aliphatic radical having at least 5 carbon atoms or an aromatic, aromatic-aliphatic, cycloaliphatic, cycloaliphatic-aliphatic, heterocyclic or heterocyclic-aliphatic radical,

and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,

and the remaining substituents are as defined above.

Preference is given especially to COMPOUNDS OF THE INVENTION wherein

R₁ is pyridyl or N-oxido-pyridyl each of which is bonded at a carbon atom,

R₂ and R₃ are each hydrogen,

R₄ is hydrogen or lower alkyl,

R₅ is hydrogen, lower alkyl or trifluoromethyl,

R₆ is hydrogen,

R₇ is nitro, fluoro-substituted lower alkoxy or a radical of formula II wherein R₉ is hydrogen, X is oxo, n is 0 and R₁₀ is pyridyl bonded at a carbon atom, phenyl that is unsubstituted or substituted by halogen, cyano, lower alkoxy, carboxy, lower alkyl or by 4-methyl-piperazinyl-methyl, or C₅-C₇alkyl, thienyl, 2-naphthyl or cyclohexyl, and

R₈ is hydrogen.

Special preference is given to COMPOUNDS OF THE INVENTION wherein at least one of the radicals R₄ and R₈ is lower alkyl, and the remaining substituents are as defined above.

Preference is given above all to COMPOUNDS OF THE INVENTION wherein

R₁ is pyridyl bonded at a carbon atom,

R₂, R₃, R₅, R₆ and R₈ are each hydrogen,

R₄ is lower alkyl,

R₇ a radical of formula II wherein R₉ is hydrogen, X is oxo, n is 0 and R₁₀ is 4-methyl-piperazinyl-methyl.

Preference is given above all especially to the COMPOUND OF THE INVENTION of formula I which is CGP 57148B {N-{5-[4-(4-methyl-piperazino-methyl)-benzoylamido]-2-methylphenyl}-4-(3-pyridyl)-2-pyrimidine-amine monomesylate}.

Very preferably a COMPOUND OF THE INVENTION is used in the form of its monomesylate salt.

In one preferred embodiment of the invention the disease to be treated is selected from allergic rhinitis, allergic dermatitis, drug allergy and food allergy. In another preferred embodiment of the invention the disease to be treated is multiple sclerosis. In a further embodiment of the invention the disease to be treated is selected from angioedema, urticaria, sudden infant death syndrome and bronchopulmonary aspergillosis. Furthermore, the COMPOUNDS OF THE INVENTION can be used for the treatment of systemic mastocytosis, especially mastocytoma. The latter disease is a malignant disease with extensive metastasis, in particular in dogs. Thus, the COMPOUNDS OF THE INVENTION are particularly useful for the preparation of a medicament for treating canine mast cell neoplasms.

The COMPOUNDS OF THE INVENTION are generically and specifically disclosed in the patent applications EP 0 564 409 A1 and WO 99/03854, in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding polymorphs, e.g., crystal modifications, which are disclosed therein.

In EP 0 564 409 A1 the COMPOUNDS OF THE INVENTION are described to be useful for the therapy of cancer, thrombosis, psoriasis, fibrosis, dermatosclerosis and atheriosclerosis. In accordance with the present invention it has now been found that COMPOUNDS OF THE INVENTION surprisingly have a beneficial effect on allergic rhinitis, allergic dermatitis, drug allergy or food allergy, angioedema, urticaria, sudden infant death syndrome, bronchopulmonary aspergillosis, multiple sclerosis, and, moreover, mastocytosis, especially canine mast cell neoplasms.

In accordance with the particular findings of the invention, the present invention also provides a method of treatment of warm-blooded animals, including humans, in which an therapeutically effective dose of a COMPOUND OF THE INVENTION is administered to such a warm-blooded animal, preferably a human or a dog, very preferably a human, suffering from one of the diseases mentioned herein.

The invention relates also to a method for administering to a dog subject having canine mast cell neoplasms a COMPOUND OF THE INVENTION or a pharmaceutically acceptable salt thereof, which comprises administering a pharmaceutically effective amount of a COMPOUND OF THE INVENTION or a pharmaceutically acceptable salt thereof to the dog once daily for preferably a period exceeding 1, 2 or even 3 months. The invention relates especially to such method wherein a daily dose of about 20-200 mg, preferably 80-160 mg, especially 125 mg of SALT I is administered.

Preferably, the invention relates to a use or method wherein a daily dose of a monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered to a dog, and said daily dose comprises an amount of said monomethanesulfonate salt sufficient to maintain plasma levels of at least 0.2 μM.

The term “method of treatment” as used herein relates especially also to a method of prevention of the diseases mentioned herein, i.e., the prophylactic administration of a pharmaceutical composition comprising a COMPOUND OF THE INVENTION to healthy patients to prevent the outbreak of the diseases mentioned herein.

The present invention relates also to a pharmaceutical composition for the treatment of at least one of the diseases mentioned herein comprising a COMPOUND OF THE INVENTION. Pharmaceutical compositions for the treatment of at least one of the diseases mentioned herein comprise an effective amount of the COMPOUNDS OF THE INVENTION together with pharmaceutically acceptable carriers that are suitable for topical, enteral, for example oral or rectal, or parenteral administration, and may be inorganic or organic, solid or liquid. For oral administration there are used especially tablets or gelatin capsules comprising the COMPOUNDS OF THE INVENTION together with diluents, and/or lubricants, for example, silicic acid, talc, stearic acid or salts thereof, and/or polyethylene glycol, but also lotions, gels or creams. Tablets may comprise binders, starches, gelatin, methylcellulose, sodium carboxymethylcellulose and/or polyvinylpyrrolidone, disintegrators, and/or effervescent mixtures, or adsorbents, dyes, flavourings and sweeteners. The COMPOUNDS OF THE INVENTION can also be used in the form of parenterally administrable compositions or in the form of infusion solutions. Topical administration is, e.g., to the skin. A further form of topical administration is to the eye, e.g., for the treatment of vernal conjunctivitis. The pharmaceutical compositions may be sterilised and/or may comprise excipients, for example, preservatives, stabilisers, wetting agents and/or emulsifiers, solubilisers, salts for regulating the osmotic pressure and/or buffers. The present pharmaceutical compositions are prepared in a manner known per se, for example, by means of conventional mixing, granulating, confectioning, dissolving or lyophilising processes, and comprise approximately from 1-100%, especially from approximately 1% to approximately 20%, active ingredient(s).

The COMPOUNDS OF THE INVENTION can, for example, be formulated as disclosed in Examples 4 and 6 of WO 99/03854.

The dosage range of the COMPOUNDS OF THE INVENTION to be employed depends upon factors known to the person skilled in the art including species of the warm-blooded animal, body weight and age, the mode of administration, the particular substance to be employed and the disease to be treated. Unless stated otherwise herein, the COMPOUNDS OF THE INVENTION are preferably administered from one to four times per day or immediately when an allergic reaction is observed. Furthermore, the COMPOUNDS OF THE INVENTION, especially N-{5-[4-(4-methyl-piperazino-methyl)-benzoylamido]-2-methylphenyl}-4-(3-pyridyl)-2-pyrimidine-amine (STI571B), are preferably administered to a warm-blooded animal, especially a human in a dosage in the range of about 10-750 mg/day, preferably 30-600 mg/day more preferably 30-300 mg/day.

In dogs, depending on species, age, individual condition, mode of administration, and the clinical picture in question, effective doses, for example, daily doses of about 20-200 mg, preferably 80-160 mg, especially 125 mg, are administered to warm-blooded animals of about 5 kg bodyweight. For adult dogs of about 5 kg with unresectable and/or metastatic malignant canine mast cell neoplasms, a starting dose of 125 mg daily can be recommended. For dogs with an inadequate response after an assessment of response to therapy with 125 mg daily, dose escalation can be safely considered and dogs may be treated as long as they benefit from treatment and in the absence of limiting toxicities. Dosages may be titered so as to achieve plasma levels of at least 0.2 μM (micromolar), preferably at least 0.5 μM, more preferably at least 1 μM. Achieving and/or maintaining a plasma level of about 1 μM is particularly preferred.

What is claimed is:

1. Use of a N-phenyl-2-pyrimidine-amine derivative of the formula I 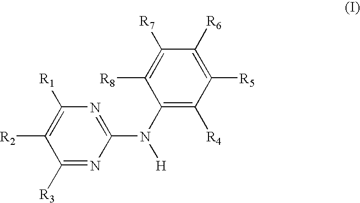
wherein
R₁ is 4-pyrazinyl; 1-methyl-1H-pyrrolyl; amino- or amino-lower alkyl-substituted phenyl, wherein the amino group in each case is free, alkylated or acylated; 1H-indolyl or 1H-imidazolyl bonded at a five-membered ring carbon atom; or unsubstituted or lower alkyl-substituted pyridyl bonded at a ring carbon atom and unsubstituted or substituted at the nitrogen atom by oxygen;
R₂ and R₃ are each independently of the other hydrogen or lower alkyl;
one or two of the radicals R₄, R₅, R₆, R₇ and R₈ are each nitro, fluoro-substituted lower alkoxy or a radical of formula II
—N(R₉)—C(═X)—(Y)ₙ—R₁₀ (II),
wherein
R₉ is hydrogen or lower alkyl,
X is oxo, thio, imino, N-lower alkyl-imino, hydroximino or O-lower alkyl-hydroximino,
Y is oxygen or the group NH,
n is 0 or 1 and
R₁₀ is an aliphatic radical having at least 5 carbon atoms, or an aromatic, aromatic-aliphatic, cycloaliphatic, cycloaliphatic-aliphatic, heterocyclic or heterocyclic-aliphatic radical,
and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,
or a salt of such a compound having at least one salt-forming group,
for the manufacture of a medicament for treating allergic rhinitis, allergic dermatitis, drug allergy or food allergy, angioedema, urticaria, sudden infant death syndrome, bronchopulmonary aspergillosis, mastocytosis or multiple sclerosis.

2. Use of a compound of formula I according to claim 1, wherein one or two of the radicals R₄, R₅, R₆, R₇ and R₈ are each nitro or a radical of formula II
wherein
R₉ is hydrogen or lower alkyl,
X is oxo, thio, imino, N-lower alkyl-imino, hydroximino or O-lower alkyl-hydroximino,
Y is oxygen or the group NH,
n is 0 or 1 and
R₁₀ is an aliphatic radical having at least 5 carbon atoms or an aromatic, aromatic-aliphatic, cycloaliphatic, cycloaliphatic-aliphatic, heterocyclic or heterocyclic-aliphatic radical,
and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,
and the remaining substituents are as defined in claim 1,
or a pharmaceutically acceptable salt of such a compound having at least one salt-forming group.

3. Use of a compound of formula I according to claim 1, wherein
R₁ is pyridyl bonded at a carbon atom,
R₂, R₃, R₅, R₇ and R₈ are each hydrogen,
R₄ is lower alkyl,
R₇ a radical of formula II wherein
R₉ is hydrogen,
X is oxo,
n is 0 and
R₁₀ is 4-methyl-piperazinyl-methyl,
or a pharmaceutically acceptable salt thereof.

4. Use of a compound of formula I according to claim 1 which compound is N-{5-[4-(4-methyl-piperazino-methyl)-benzoylamido]-2-methylphenyl}-4-(3-pyridyl)-2-pyrimidine-amine, or a pharmaceutically acceptable salt thereof.

5. Use of a compound of formula I according to any one of claims 1 to 4, wherein the compound is used in the form of its monomesylate salt.

6. Use of a compound of formula I according to any one of claims 1 to 5, for the manufacture of a medicament for treating canine mast cell neoplasms.

7. Method of treatment of a warm-blooded animal having allergic rhinitis, allergic dermatitis, drug allergy or food allergy, angioedema, urticaria, sudden infant death syndrome, bronchopulmonary aspergillosis, mastocytosis, or multiple sclerosis comprising administering to the animal a compound of formula I as defined in any one of claims 1 to 5 in a quantity which is therapeutically effective against at least one of the diseases mentioned above in which a compound of formula I can also be present in the form of a pharmaceutically acceptable salt.

8. A method of treating dogs suffering from canine mast cell neoplasms which comprises administering to a said dog in need of such treatment a dose, effective against canine mast cell neoplasms, of a compound of formula I as defined in any one of claims 1 to 5.

9. Use or method according to claims 8 or 6 wherein a daily dose of 20-200 mg of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof is administered to an adult dog.

10. Use or method according to claims 1 or 7 wherein a daily dose of 30-400 mg of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof is administered to a human.
